(19) United States
(12) Patent Application Publication
(10) Pub. No.: US 2006/0100666 A1
(43) Pub. Date: May 11, 2006

(54) Apparatus and method for lung analysis

(75) Inventors: Malcolm Howard Wilkinson, Forest Hill (AU); Clive Andrew Ramsden, Cheltenham (AU); Philip John Berger, Carlton (AU)

Correspondence Address: LADAS & PARRY, 26 WEST 61ST STREET, NEW YORK, NY, 10023, US

(73) Assignee: PULMOSONIX PTY. LTD.

(21) Appl. No.: 11/111,689

(22) Filed: Apr. 21, 2005

Related U.S. Application Data
(63) Continuation-in-part of application No. 10/272,494, filed on Oct. 15, 2002.
Application No. 10/272,494 is a continuation of application No. PCT/AU01/00465, filed on Apr. 20, 2001.

(30) Foreign Application Priority Data
Jun. 2, 2004 (AU) 2004902932
Apr. 20, 2000 (AU) PQ7040
Apr. 10, 2001 (AU) PR 4333

(51) Int. Cl. A61N 1/39

(52) U.S. Cl. 607/1

(57) ABSTRACT

An apparatus and method of detecting COPD and in particular, emphysema utilizes a change in acoustic transmission characteristics of a lung due to e.g. the appearance of fenestrae (perforations) in the alveoli of the lung. The use of acoustic signals may provide good sensitivity to the existence of alveolar fenestrae, even for microscopic emphysema, and the appearance and increase in fenestrae may be determined by monitoring acoustic transmission characteristics such as, for example, an increase in acoustic signal velocity and velocity dispersion, and/or a change in attenuation. A transmitter may be located in e.g. the supra-clavicular space and receivers may be mounted on the chest. Measurements may be correlated between pairs of receivers to determine acoustic transmission profiles.

The present application is a continuation-in-part of U.S. Ser. No. 10/272,494 entitled “Method and Apparatus for Determining Conditions of Biological Tissues” filed on 15 Oct. 2002, which is a continuation of International Patent Application No. PCT/AU01/00465 entitled “Method and Apparatus for Determining Conditions of Biological Tissues” filed on 20 Apr. 2001. The present application also claims priority from Australian provisional patent application No. 2004902932 entitled “Apparatus and Method for Lung Analysis” filed on 2 Jun. 2004. The contents of each of these applications are incorporated by reference in their entirety in the present application.

The present invention relates to a method of determining characteristics of biological tissues in humans and animals. In particular, it relates to determining the characteristics of tissues such as the lungs and airways by introducing a sound to the tissue, and measuring one or more characteristics of the sound. The invention further includes an apparatus capable of such measurement. The invention relates in particular to methods and apparatus for detecting Chronic Obstructive Pulmonary Disease (COPD) and more particularly, emphysema.

### BACKGROUND TO THE INVENTION

Non-invasive determination of the condition of biological tissues is useful in particular where the patient is unable to co-operate or the tissue is inaccessible for easy monitoring.

Techniques presently used in determining the characteristics of biological tissues include x-rays, magnetic resonance imaging (MRI) and radio-isotopic imaging. These are generally expensive and involve some degree of risk which is usually associated with the use of x-rays, radioactive materials or gamma-ray emission. Furthermore, these techniques are generally complicated and require equipment which is bulky and expensive to install and, in most cases, cannot be taken to the bedside to assess biological tissues in patients whose illness prevents them being moved.

Sound waves, particularly in the ultra-sound range have been used to monitor and observe the condition of patients or of selected tissues, such as the placenta or fetus. However, the process requires sophisticated and sometimes expensive technology and cannot be used in tissues in which there is a substantial quantity of gas, such as the lung.

The lungs supply oxygen to, and remove carbon dioxide from, the blood. Air enters the lungs via the trachea and the bronchial tube of each lung. The two bronchial tubes branch into secondary bronchi that form the lobes of the lung, and these secondary bronchi further branch to form numerous smaller tubes (bronchioles) that terminate in small gas-exchanging air sacs called alveoli. A network of capillaries runs through the walls of the alveoli, and oxygen and carbon dioxide are exchanged across these walls between the air in the alveoli and the blood in the capillaries.

Every year in Australia about 5000 newborn infants require, a period of intensive care (ANN Annual Report, 1996-1997). Respiratory failure is the most common problem requiring support and is usually treated with a period of mechanical ventilation. Over the last decade the mortality of infants suffering respiratory failure has shown an impressive decline, attributable at least in part to improved techniques used in mechanical ventilation, and the introduction of surfactant replacement therapy (Jibe, 1993). The vast majority of infants now survive initial acute respiratory illness, but lung injury associated with mechanical ventilation causes many infants to develop ‘chronic lung disease’.

Chronic lung disease is characterized by persisting inflammatory and fibrotic changes, and causes over 90% of surviving infants born at less than 28 weeks gestation, and 30% of those of 28-31 weeks gestation, to be dependent on supplementary oxygen at 28 days of age. Of these, over half still require supplementary oxygen when they have reached a post-menstrual age of 36 weeks gestation (ANZNN Annual report, 1996-1997). Assistance with continuous positive airway pressure (CPAP) or artificial ventilation is also commonly required.

Historically, barotrauma and oxygen toxicity have been considered to be the primary culprits in the etiology of chronic lung disease (Northway et al, 1967; Taghizadeh & Reynolds, 1976). However, trials of new strategies in mechanical ventilation which were expected to reduce barotrauma and/or exposure to oxygen have often had disappointingly little impact on the incidence of chronic lung disease (HIFI Study Group,1989; Bernstein et al, 1996; Baumer, 2000). Comparison of strategies of conventional mechanical ventilation in animals (Dreyfuss et al, 1985) have indicated that high lung volumes may be more damaging than high intrapulmonary pressures, and has led to the concept of ‘volutrauma’ due to over-inflation of the lung. At the same time, experience with high frequency oscillatory ventilation (HFOV) has indicated that avoidance of under-inflation may be equally important.

HFOV offers the potential to reduce lung injury by employing exceptionally small tidal volumes which are delivered at a very high frequency. However, this technique fails to confer benefit, if the average lung volume is low (HIFI Study Group, 1989), yet it appears to be successful if a normal volume is maintained (McCulloch et al, 1988; Gerstmann et al, 1996). This highlights the importance of keeping the atelectasis-prone lung ‘open’ (Froese, 1989). Evidence of this kind has led to the concept that a ‘safe window’ of lung volume exists within which the likelihood of lung injury can be minimized. The key to preventing lung injury may lie in maintaining lung volume within that safe window thereby avoiding either repetitive over-inflation or sustained atelectasis. (See FIG. 1).

Even when lung volume is maintained in the “safe window”, changes in the lung condition may manifest due to the general damaged or underdeveloped condition of the lung. Fluid and blood may accumulate in the lung, posing additional threats to the patient. Evaluation with a stethoscope of audible sounds which originate from within the lung (breath sounds) or are introduced into the lung (by percussion, or as vocal sounds) forms an essential part of any routine medical examination. However, in the sick newborn, the infant's small size, inability to co-operate and the presence of background noise greatly limits the value of such techniques. The value of these techniques is further limited by the lack of reproducibility of the sounds and the subjective nature of the analysis which follows.

Also relating to the condition of the lung, Chronic Obstructive Pulmonary Disease (COPD) is the leading cause of respiratory deaths worldwide. COPD places enormous economic burden on society. Medical expenses for COPD patients are extremely high because of frequent visits to the emergency room, extended hospital stays and expensive medications. In developed countries, the major cause of COPD is cigarette smoking but two distinct and overlapping diseases (together called COPD) result: chronic bronchitis and emphysema.

Chronic bronchitis is a neutrophil led chronic inflammatory airways disease with regular exacerbations leading to true narrowing of airways and increased resistive work of breathing. The key elements of therapy are the removal of the toxic stimulus (i.e. Stocking cessation), bronchodilator therapy, anti-inflammatory drugs, mucolytics, prevention and early treatment of infection as well as rehabilitation.

In emphysema, the problems are distinctly different. The lung parenchyma is destroyed with a reduction in gas exchanging area. Dynamic collapse of untethered airways occurs leading to increased expiratory work of breathing and gas trapping of the lung. This gas trapping makes the lung work at a higher lung volume (at which it is stiffer), increasing inspiratory work of breathing. The over-distension also markedly reduces the mechanical efficiency of the diaphragm. Exercise is terminated early because of rapidly rising and unsustainable work of breathing.

Smoking cessation and prevention of infection are the keys to prevent disease progression. Bronchodilators and anti-inflammatory drugs would be predicted to have little benefit but rehabilitation is of proven benefit. In more advanced disease interventions to improve the mechanical properties of the lung, for example lung volume reduction surgery and highly novel minimally invasive approaches, as well as transplantation in a few, are the most likely to significantly improve functional capacity.

Emphysema is a slowly progressive disease of the lung. It involves the gradual destruction of the alveolar walls. The loss of alveolar tissue results in a loss of gas exchange surface area and decreases the number of capillaries available for gas exchange. It also reduces the elastic recoil of the lung and leads to the collapse of the bronchioles and chronic airflow obstruction. Thus, lung function is gradually lost through a reduction in gas-exchange area and in the amount of air that reaches the alveoli.

Emphysema afflicts millions of people worldwide. Statistics show that in 2002 over three million people were affected by the disease in the US alone, 50% being over 65 years of age. By 2020, emphysema and obstructive airway disease are expected to be the third leading cause of death after cancer and heart disease. Although the exact causes of the disease are not understood, smoking is a major factor, with an estimated 20% of smokers contracting the disease at some time in their lives.

Current methods of emphysema detection include MRI high resolution CAT scans and spirometry. These methods are however somewhat complex and expensive, and are not well suited to the rapid screening of people at risk. Lung function testing can also be used to identify obstructive airway disease associated with emphysema, but this can only identify the advanced stages of the disease, by which time there has already been widespread and irreversible damage.

Presently the way physicians try to differentiate between chronic bronchitis and emphysema is using the diffusing capacity (which lacks specificity) and high resolution CT scan (which lacks sensitivity). Thus unless advanced emphysema is present, the usual approach is that if the diffusion capacity is substantially reduced physicians suggest the likelihood of emphysema. In large clinical (drug) studies COPD is studied as a single group which means the overall effect of the therapy tried is very small leading to very high numbers of patients treated to achieve the endpoints measured.

Whilst determining and monitoring lung condition in newborn babies is difficult, determining lung condition in adults can be equally as challenging, particularly if a patient is unconscious or unable to cooperate. This places a further limitation on the presently available techniques for monitoring lung condition.

It would thus be desirable to be able to perform early detection of emphysema, convincing smokers to stop smoking before serious permanent damage occurs. A more precise diagnosis allowing physicians to stop treating emphysema patients with the same approach as chronic bronchitis patients would also be desirable, as would better stratification in drug studies and ultimately in clinical practice. This would provide cost savings of hundreds of millions of dollars.

The present invention aims to provide new and advantageous apparatus and methods for assessing and detecting COPD and in particular, COPD in the form of emphysema.

### SUMMARY OF THE INVENTION

Viewed from one aspect, the present invention provides a method for determining the presence of Chronic Obstructive Pulmonary Disease (COPD) in a lung. The method includes the steps of applying an acoustic signal to the lung and detecting the signal after it has passed through at least part of the lung. The method also includes determining an acoustic transmission characteristic indicative of the microstructure of the at least part of the lung. COPD is determined to be present when the acoustic transmission characteristic indicates the existence of a feature of COPD.

In determining an acoustic transmission characteristic indicative of the microstructure of the lung or a part thereof, it is to be understood that “microstructure” includes a condition of the microstructure of the lung (or part thereof) or a parameter of the same. The microstructure of the lung relates to the alveolar structure. Accordingly, conditions and parameters thereof include, but are not limited to, the presence of fenestrae in the alveoli, the number and/or size of such fenestrae and the presence of fluid, inflammation and scarring in the microstructure of the lung or a part thereof.

Thus in one embodiment, the invention determines the presence of COPD by determining whether the acoustic transmission characteristic indicates the existence of features of COPD in the lung, such as fenestrae in the alveoli which is indicative of emphysema. The term “acoustic signal” should be taken to relate to sound waves, i.e. signals in the audible frequency range, e.g. of a frequency from about 20 Hz to about 25 KHz.

Thus, the present invention may be seen as sensing acoustic transmission properties in the lung that relate to the existence of features of COPD and in particular, changes in the microstructure of the lung, such as the development of alveolar fenestrae. The present invention is able to provide a non-invasive, relatively inexpensive, quick and easily implemented test for COPD. It may be especially useful in a COPD (and in particular, emphysema) screening program, and in testing a subject who is unable to co-operate with examination such as an infirm or incapacitated subject.

The present invention may also be used to screen asymptomatic subjects to determine the likelihood of a subject going on to develop COPD and in particular, emphysema. Such screening is particularly well suited to large-scale screening of asymptomatic smokers to determine which of those smokers exhibit a propensity to developing COPD and in particular, emphysema. This can be achieved by screening for small changes in the lung's microstructure, recognizable by small changes in acoustic signal velocity, velocity dispersion, attenuation, attenuation dispersion, and/or power density for a single frequency or a range of frequencies. Those smokers who exhibit small changes in the lung's microstructure such as an increase in the number of alveolar fenestrae would be considered more likely to develop advanced stages of COPD and in particular, emphysema.

The present invention may also be used as a staging tool, to determine a stage of development of disease (e.g. early stage, mid stage or advanced). In this regard, one may screen for progressively larger changes in signal velocity, velocity dispersion, attenuation, attenuation dispersion, and/or power density for a single frequency or a range of frequencies.

In one embodiment, the present invention exploits the effect that fenestrae (perforations) in the alveoli of a lung have on the acoustic transmission characteristics of the signal to indicate the onset of emphysema and the progression of the disease. It recognizes that changes in the microstructure or alveolar structure of the lung caused by an increase in the number of fenestrae or pores connecting neighboring alveoli, and which may be seen as a movement from a closed-cell type arrangement to an open-cell type arrangement, will cause a measurable and identifiable change in the acoustic transmission properties of the lung.

This change in the “cellular structure” of the lung (i.e. open vs. closed) has the effect of changing the acoustic permeability of the lung tissue, which can be detected by monitoring the acoustic transmission characteristics of the lung. At least in the emphysematous lung, the changes in cell-type occur in very early stages when the patient may still be asymptomatic and before there is any noticeable change in the lung density. Accordingly, the present invention has the capability to detect early stages of COPD and particularly emphysema which cannot be detected using the existing lung analysis methods.

Accordingly, embodiments of the present invention may be especially useful in diagnosing emphysema in its early stages, e.g. the existence of microscopic emphysema. From another aspect, the present invention may be seen as a recognition that measurable acoustic transmission property changes may be associated with such microscopic fenestrae. These fenestrae may for example be of the order of 10 or 20 microns or more in diameter. Early stage diagnosis of emphysema is especially useful because emphysema damage is irreversible, and so the earlier it is diagnosed the earlier a treatment regime, including cessation of smoking and rehabilitation may be commenced to minimize further progression and effects of the disease.

In one embodiment of the present invention, signal velocity through the lung is detected, and a determination is made as to whether one or more of the detected velocity characteristics are indicative of perforated/fenestrated alveoli. Thus, a determination may be made as to whether the velocity of an acoustic signal through a lung is greater than a signal velocity associated with a normal lung. The magnitude of the signal velocity may be used to indicate the stage of emphysema, as may changes in velocity which are detected as the signal propagates through the lung.

The signal velocity may be determined for a single acoustic frequency, or for a range of frequencies. In the latter case, emphysema may be determined based on a characteristic of the velocity profile over a range of frequencies or an average of the velocities. In one embodiment, the velocity dispersion may be determined. Thus, generally for a normal or diseased lung, signal velocity will vary based on signal frequency. In accordance with embodiments of the present invention, an increase in velocity dispersion, i.e. a larger spread of velocities for a particular frequency range (or put another way a larger change in velocity for a particular frequency range) may indicate existence of COPD features such as alveolar fenestrae which are indicative of emphysema. The amount of dispersion may indicate the degree of COPD/emphysema or stage thereof.

In one embodiment, signal attenuation through the lung is detected, and a determination is made as to whether one or more of the detected attenuation characteristics are indicative of a feature of COPD such as, for example, perforated alveoli, inflammation of the airways, bronchorestriction, or increased mucus production in the airways. Thus, a determination may be made as to whether the attenuation of an acoustic signal through a lung is different from signal attenuation associated with a normal lung and indicative of COPD. The amount of the signal attenuation may be used to indicate the degree of COPD/emphysema and/or provide an indication as to the stage of development of the disease.

Attenuation may be determined for a single frequency, or for a range of frequencies. In the latter case, COPD may be determined based on a characteristic of the attenuation profile over a range of frequencies, or an average of the attenuation. In one embodiment, the frequency dependence of attenuation may be determined. Thus, generally for a normal or diseased lung, the signal attenuation will vary based on signal frequency, for example, a change in attenuation may be more noticeable for lower frequency sounds. In accordance with the present invention, a larger change in attenuation at certain frequencies may be used to indicate existence of features of COPD. The magnitude of this change may indicate the degree of COPD.

It is to be understood that a combination of two or more of the above characteristics may be used to assess the existence of emphysema. For example, the velocities of one or more of the acoustic frequencies and the velocity dispersion of the acoustic signal may both be used to assess emphysema. Similarly, a combination of two or more of the above characteristics may be used to assess the existence of chronic bronchitis or other forms of COPD. For instance, the attenuation of one or more of the acoustic frequencies and the attenuation dispersion of the acoustic signal may both be used to assess the existence of chronic bronchitis. Other acoustic characteristics such as signal power density may be used as an alternative or in addition to the above.

The acoustic signal may be applied in any suitable manner, and may be detected after transmission through the whole width or length of a lung or after only having traveled through part of the lung. For example, the signal may be applied via the trachea through a mouthpiece. Preferably, however, the signal is applied trans-thoracically. That is, the signal is transmitted from one part of the thorax and detected at another part of the thorax. The acoustic signal may for example be applied dorsally and detected ventrally. Thus, a transmitter may be placed in contact with a subject's back and a receiver may be placed on the subject's torso. The signal may be applied at any level of the lung, e.g. at the top, middle or bottom. Alternatively, the signal may be applied ventrally and detected dorsally.

In one preferred embodiment, the acoustic signal is applied in the region of the supra-clavicular space. It has been found that the application of the signal at this location has a number of advantages. In one aspect, the detected signal can be cleaner, in that unwanted reflections of sound waves from adjacent solid structures may be reduced as compared with a dorsal-ventral transmission. From another aspect, the supra-clavicular space is adjacent to the apex of the lung. The upper-lobes of the lung are commonly affected in emphysema. Accordingly, injection of the sound at the apex of the lung where the disease is likely to be present may minimize the effects of acoustic transmission characteristics associated with other regions of the lung which are of less interest (not likely to be affected by the disease). Appropriate positioning of receivers would assist in this regard.

The transmitted acoustic signal may be detected at any suitable point remote from the application point. In one embodiment, a receiver may be placed on the chest of the patient at the level of the base of the upper lobe of the lung on the side being examined. Alternatively, additionally, one or more receivers may be placed on the patient's back or side.

In another embodiment, two or more receivers are used. For example, if the signal is applied dorsal-to-ventral, then receivers may be placed at a number of locations on the chest, and each of the receivers may provide an indication of lung condition in a region of the lung between the transmitter and that receiver. This may help to indicate the progress of the disease through the lung, to create a “disease map” of the lung, or assist in staging the disease. For example, a set of vertically placed receivers may be used to indicate the degree to which the disease has progressed down the lung, and/or may be used to test the different lung lobes. Alternatively or additionally, a plurality receivers may be located in an array near the dorsal spine and detect a plurality of signals which can be used to determine the acoustic transmission characteristics of different regions of the lung.

In the case of a signal applied at the supra-clavicular space, receivers may be placed in-line with the transmitter on the chest of the subject. Again, the signals from each of the receivers may be used to determine the progress of COPD through the lung. Also, when the receivers are placed progressively further from the transmitter, the signals between the receivers, rather than between the transmitter and each receiver, may be compared in order to determine the acoustic transmission characteristics between the receivers. This may be particularly advantageous in situations where it is more difficult to determine precisely the distance between the transmitter and a receiver, than the distance between two receivers which can be set to a known value.

Thus, one embodiment may consist of a transmitter and two or more receivers, wherein the acoustic transmission characteristics of the lung are determined from a comparison of the signals detected at the receivers. The receivers may be in-line or they may be in a scattered/array arrangement.

The transmitter itself may take any suitable form capable of generating acoustic signals. One suitable transmitter is in the form of an electro-acoustic transducer that may, for example, couple directly to the surface of the thorax or may be coupled to the surface of the thorax via an air-chamber. The transmitter may be attachable to a patient or handheld, and be shaped so as to sit within the super-clavicular space of the subject under examination. The receiver or receivers may take any suitable form. They may for example be microphones such as simple electret microphones, hydrophones, or accelerometers.

The acoustic signal may also take any suitable form. It is preferably of a form that is able to be well distinguished from environmental noises such as breath sounds, coughs and wheezes. It may comprise a single pure tone (monophonic) that may be pulsed, and may include a plurality of such tones (polyphonic), each of different frequency and pulsed one after the other or simultaneously. Velocity and/or attenuation could then be determined for each frequency individually. Alternatively, the acoustic signal comprises pseudorandom noise. Preferably, the transmitted and received signals or the signals between two receivers are cross-correlated to provide a high degree of rejection of extraneous disturbances from the signal. The cross-correlation can also be used to establish an impulse response for the system. A frequency domain transfer function may be obtained, e.g. by taking the Fast-Fourier Transform (FFT) of the impulse response, from which required transmission characteristics, for example velocity, attenuation and dispersion, may be determined.

The acoustic signal should have sufficient amplitude to produce an acceptable signal-to-noise ratio. An example of a suitable sound pressure level applied to the thorax is 120 decibels or approximately 20 Pascals though other levels may also be suitable. It should be noted that, since the signal is applied directly to a small area of the body, high decibel signals can be used without discomfort, as the transducer is sufficiently shielded that the sound is barely audible to the subject.

The acoustic signal may include frequencies in the range of 70 Hz to 5 kHz, as these frequencies have been found to produce very good results. In one embodiment, frequencies lower than 1 kHz are used.

The transmission characteristics of the lung may also be affected by the amount of inflation of the lung, i.e. the gas fraction of the lung. In one embodiment, therefore, the signal transmission characteristics are determined at a particular lung inflation. In one embodiment, the measurements are made at functional residual capacity, i.e. at a lung volume when the muscles of the chest wall and diaphragm and abdomen are relaxed. Measurements could also be made at maximum inspiration since there is evidence to suggest that at high lung volumes the size of fenestrae may increase, rendering them more detectable.

A point to note is that in accordance with another aspect of the present invention, the emphysematous lung can be acoustically modeled as a system of interacting alveoli with an open-cell structure with multiple pores interconnecting each of the alveoli, rather than a system of isolated alveoli with an essentially closed cell structure as in the normal lung. In this model, energy dissipation of the signals may be determined through viscous loss as the sound waves pass through the fenestrae that connect neighboring alveoli, rather than by absorption through resonating isolated alveoli. This model produces a frequency dependency for the attenuation that is not as great as for the traditional closed cell model of the normal lung. Moreover, this model has the unique feature of contemplating porosity of the lung, and microscopic changes in porosity which occur with COPD development, rather than contemplating lung density.

Viewed from another aspect, the present invention provides apparatus for determining the presence of Chronic Obstructive Pulmonary Disease (COPD) in a lung. The apparatus includes a transmitter for transmitting an acoustic signal into a lung and one or more receivers for detecting the acoustic signal after it has passed through at least part of the lung. A controller/processor is also provided for determining an acoustic transmission characteristic indicative of the microstructure of the at least part of the lung and for determining whether the transmission characteristic indicates the presence of a feature of COPD.

In one embodiment, the apparatus further includes a sheath and the transmitter and plurality of receivers are retained on the sheath. In such an embodiment, the sheath can be worn by a subject during use of the apparatus, and individual attachment of the transmitter/receiver(s) to the subjects torso is obviated. This has the advantage of reliable and reproducible transducer positioning and, depending on the nature of the sheath, may improve the coupling between the transducers and the subject's torso.

In one arrangement, the sheath is a vest worn by the subject. In another arrangement, the sheath is filled with a fluid such as water, saline or gel-like solution and contains hydrophones positioned in the fluid. The hydrophones may provide better coupling, and reduce the influence of the ribs on the detected sound signal. Hydrophones may also provide a much wider frequency response and better noise shielding than other types of receivers. The receiver(s) may be located ventrally, or dorsally. In one embodiment, a plurality of receivers are located on the dorsal spine. Such receivers may be provided in pairs located longitudinally along the dorsal spine. Alternatively, they may be provided in an array centered on the spine. Alternatively/additionally, one or more receivers may be located on the subject's torso and/or below the armpit.

The apparatus may further include a display device for displaying information relating to factors determined using the apparatus. Such factors may include one or more of a presence of COPD in the lung, a presence of COPD in the lung in the form of emphysema, a likelihood of developing COPD in the lung, a likelihood of developing COPD in the lung in the form of emphysema, suitability of a subject for participating in an emphysema drug trial, suitability of a subject for treatment with an emphysema drug, stage of COPD in a subject, stage of COPD in a subject, the COPD being in the form of emphysema and a map of the existence of COPD in a subject's lung.

Viewed from a further aspect, the present invention provides a method of determining chronic obstructive pulmonary disease (COPD), including the steps of: applying an acoustic signal to the lung, measuring the signal after it has passed through the lung, determining an acoustic transmission characteristic of the lung, and determining the presence of chronic obstructive pulmonary disease by determining whether the acoustic transmission characteristic indicates the existence of COPD features, e.g. indicate fenestrae in the alveoli.

Viewed from still a further aspect, the present invention provides a method of determining emphysema based on an acoustic transmission characteristic of the lung which assumes that the lung has a degree of open-cell structure in which the alveolar surface is punctured by fenestrae. For example, the method includes the step of modeling the lung to have a degree of open-cell structure, and determining an acoustic transmission characteristic associated with such a structure.

In another aspect of the present invention there is provided a method of determining characteristics of biological tissue in situ. The method includes introducing a sound to the tissue at first position, detecting the sound at another position spaced from the first position after it has traveled through the tissue, calculating the velocity and attenuation of sound that has traveled through the tissue from the first position to another position and correlating the velocity and attenuation of the detected sound to characteristics of the biological tissue.

It should be noted that any one of the aspects mentioned above may include any of the features mentioned in relation to any of the other aspects mentioned above, as appropriate.

### BRIEF DESCRIPTION OF THE FIGURES

Embodiments of the present invention will now be described, by way of example only, with reference to the accompanying drawings. It is to be understood that the particularity of the drawings does not supersede the generality of the preceding description of the invention.

FIG. 1 shows a pressure-volume curve of a moderately diseased lung illustrating two hazardous regions of lung volume, and indicating an optimal “safe” window there between (from Froese, 1997).

FIG. 2 shows a block diagram indicating steps involved in performing an embodiment of the inventive method.

FIG. 3 shows a schematic diagram of an apparatus for detecting emphysema connected to a subject to be examined;

FIGS. 4a and 4b show an arrangement of a plurality of receivers located ventrally and dorsally respectively and configured to detect an acoustic signal transmitted transthoracically.

FIG. 5 shows a schematic diagram indicating the theory behind the detection method of an embodiment of the present invention;

FIG. 6 is a graph of signal frequency against velocity through a lung for various sizes of fenestrae; and

FIG. 7 is a graph of signal frequency against velocity through the lung for a number of lung analogs exhibiting different sizes of fenestrae.

### DETAILED DESCRIPTION

Characteristics of biological tissues can be determined by measuring the velocity and attenuation of a sound as it propagates through the tissue. This can be achieved by introducing a sound to a particular location or position on the tissue, allowing the sound to propagate through the tissue and measuring the velocity and/or attenuation with which the sound travels from its source to its destination, the destination including a receiver which is spatially separated from the sound source.

It is particularly desirable that the tissue is porous comprising a composite structure made up of tissue and gas, or has regions of high and low density. Preferably the tissue is of the respiratory system. More preferably the tissue is lung tissue.

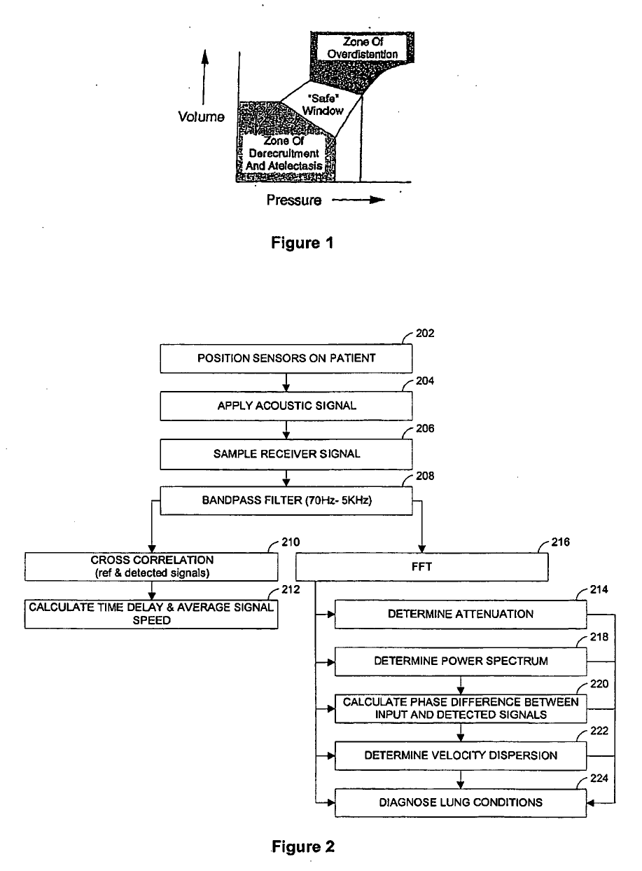

Referring to FIG. 2, in a method of determining the presence of COPD in a lung, in a step 204, an acoustic signal is applied to the lung. In a step 206, the signal is detected after it has passed through at least part of the lung. In steps 208 to 224 one or more acoustic transmission characteristics of the lung are determined. COPD is determined to be present when an acoustic transmission characteristic, indicative of the microstructure of the lung, indicates the presence of a feature of COPD.

Commercially available acoustic hardware and software packages may be used to generate a psuedo-random noise or other acoustic signal, and to perform initial data processing. External noise which is not introduced to the tissue as part of the psuedo-random noise signal is strongly suppressed by cross-correlation thereby improving the quality of the measurements made.

A separate analysis of the relative transmission of the sound through the tissue can be used to identify resonant and anti-resonant frequencies of the thorax and tissue which is being assessed. Changes in these frequencies can then be used to assess regional differences in tissue topology which may be related to pathology.

Using relationships between sound transmission velocity in tissues and the tissue characteristics themselves, it is possible to obtain a workable relationship between acoustic measurements and lung pathology.

The inventive method provides a virtually continuous real-time determination of COPD by monitoring acoustic transmission characteristics such as velocity and attenuation of a sound signal as it propagates through the lung. The method is applicable in both adults and infants, and for humans and animals. In particular, the present invention can be used in the determination of respiratory conditions in subjects who cannot co-operate with presently available conventional stethoscopic and other methods of respiratory condition analysis which require co-operation. It is also useful where the patient is critically ill, is unconscious, or is unable to respond or generate a sound which can be used to determine lung condition. Lung condition includes lung pathology such as emphysema.

The technique can be used to assist in diagnosing lung disease wherein a sound is introduced to the thorax such that it travels from one side of the thorax, through the lung, to another side of the thorax. The sound velocity and preferably attenuation which is measured is then compared with that of a normal, healthy lung. Since lung disease often manifests in reduced lung volume, a comparison can be used, again, to provide an indication as to whether a subject's lung exhibits a propensity for lung disease. Common lung diseases may include emphysema, asthma, regional collapse (atelectasis), interstitial edema and both focal lung disease (e.g. tumor) and global lung disease (e.g. emphysema). Each of these may be detectable when measurements of the velocity and attenuation of a sound which is transmitted through a diseased lung is compared with that of a lung in normal condition.

The present invention can be used to provide a monitoring system which measures sound velocity and preferably combines sound velocity data with measurements of sound attenuation. Spectral analysis of the impulse response can indicate frequency components in the sound signal which are more prominent than others and which may be an indicator of pathological or abnormal tissue.

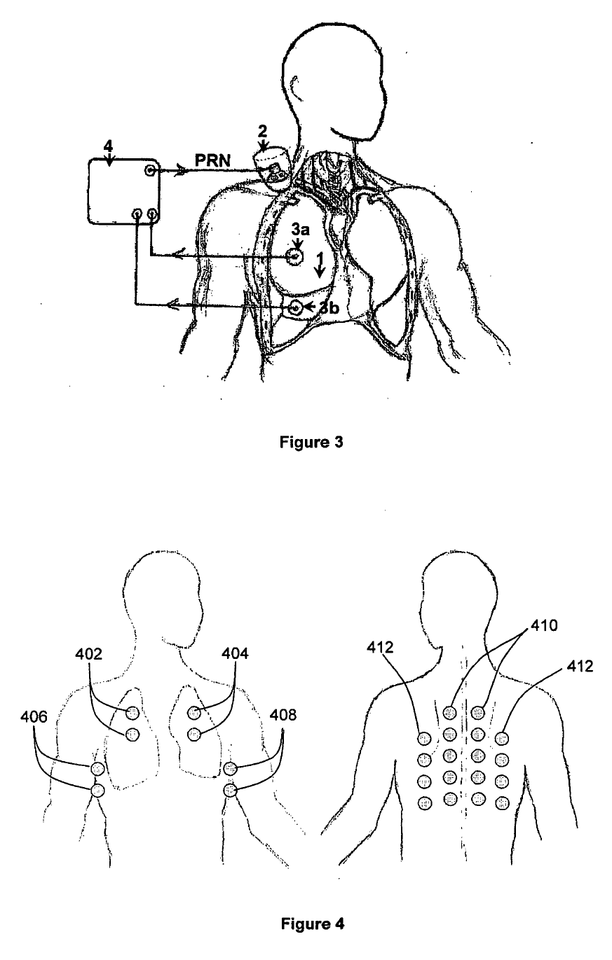

Referring to FIG. 3, one embodiment of an apparatus for detecting emphysema in a lung 1 includes a transmitter 2 for applying an acoustic signal to the lung 1, one or more spaced acoustic detectors/receivers 3a, 3b for detecting the acoustic signal after it has passed through the lung 1 or a portion thereof, and a controller 4 for controlling the transmitter 2 and for analyzing the signals detected by the receivers 3a,3b. The transmitter 2 may be, for example, an electro-acoustic transducer, and the receivers 3a,3b may be, for example, microphones such as electret microphones, accelerometers or the like.

The etiology of COPD is not well understood. However, in accordance with embodiments of the present invention, the acoustic transmission characteristics of a lung are determined, and analyzed to determine if they are indicative of a feature of COPD such as fenestrae in the alveoli, inflammation of the bronchial tubes, bronchorestriction, or increased mucus production in the airways. In emphysema, a special form of COPD, perforations (fenestrae) appear in the walls of the alveoli, changing the structure of the lung from a closed-cell type tissue structure towards an open-cell tissue structure. This causes the lung to lose elasticity and eventually leads to a collapse of associated bronchioles.

The transmitter 2 and receivers 3a,3b may be located at any suitable positions on a subject so as to determine the transmission characteristics of any desired part of the lung 1. The transmitter 2 could, for example, apply the acoustic signal through the trachea, although it is preferred to apply the signal directly to the chest cavity, e.g. by applying a signal dorsally with a transmitter positioned on the subject's back.

In the arrangement shown in FIG. 2, the transmitter 2 is positioned ventrally in the supra-clavicular space above the lung 1. This can have the advantage that the sound detected at the receivers 3a,3b is cleaner, with less noise from surrounding solid structures than might be achieved when the transmitter is positioned dorsally. Also, the sound is applied near the apex of the lung, which is where emphysema often begins. Hence detection of emphysema and potentially other forms of COPD may be more effective.

In one embodiment, receivers 3a,3b may be placed at a number of positions on the chest, to detect the sound that has been transmitted through the lung to the chest wall. The positioning may depend on the portion of the lung to be analyzed. If the signal is applied dorsal-to-ventral, the receivers may be mounted at different locations on the chest so that the characteristics of the lung may be determined for regions between the transmitter and each of the receivers.

In the example shown in FIG. 2, the receivers 3a,3b are in-line with one another and with the transmitter 2. This allows the lung characteristics between the transmitter 2 and the first receiver 3a and/or between the first receiver 3a and the second receiver 3b to be determined. Determining transmission characteristics between two receivers may be preferable to obtaining the transmission characteristics between a transmitter and a receiver, as the spatial relationship between two receivers may be more easily and more accurately obtained.

In the positions shown, the upper receiver 3a is located on the midline in the second intercostal space, and the lower receiver 3b is located at the fourth or fifth intercostal space. In this case, the acoustic transmission characteristics between these two receivers can be used to determine the presence of features of COPD in the lower lobes of the lung. The placement in one of the intercostal spaces is particularly useful, as this allows for precise location of the sensors, and also the transmission of the sound is not affected by the ribs. Placement on the midline of the lungs is also advantageous.

The receivers 3a,3b may be placed in other suitable positions also, and may be provided in any quantity. They need not necessarily be in-line with the transmitter 2. For example, in another embodiment, a receiver may be provided on each of the lung lobes. An additional receiver may also be placed in or at the location of the acoustic transducer 1, so as to monitor the sound signal that is actually input into the subject.

FIGS. 4a and 4b respectively show examples of suitable arrangements for positioning an array of receivers on the patients ventral and dorsal torso. FIG. 4a shows pairs of receivers 402, 404, 406 and 408 located ventrally on the subject's torso. Receiver pairs 402 and 404 detect acoustic transmission characteristics of the right and left lung lobes respectively, while sensors 406 and 408 detect transmission characteristics received below the patient's underarm. Receivers positioned below the underarm produce signals which are characteristic of the lung apex which is particularly well suited to the detection of emphysema which, in many cases, originates at the apex. It is to be understood that the arrangement of sensors is not to be limited to arrangement in pairs. Single receivers or groups of 3 or more may be used in one or more of the regions identified above, and in other regions.

FIG. 4b shows rows of sensors 410 placed along the dorsal spine and arranged in pairs across the spine. In the embodiment illustrated, the top sensors are placed between the shoulder blade and the spine, with the rows extending therefrom, down the spine. Such positioning provides reliable reference for future studies of that patient. In some conditions, such as gross emphysema, the lung becomes enlarged and affected regions may extend beyond the region detected by rows 410. Accordingly, additional rows of receivers, 412 may be placed on the subject's torso dorsally to detect acoustic transmission characteristics of the entire lung affected.

An advantage of dorsal placement of receivers relates to noise from the ventrally located sound source which radiates through and over the chest to ventrally located receivers. Positioning at least some of the receivers dorsally limits this effect. Hence, a clearer transmitted signal which is more representative of the acoustic permeability and acoustic transmission characteristics of the lung can be obtained.

The input sound may be, for example, a single tone or a plurality of tones emitted simultaneously or separately. They may be emitted in bursts, and their times-of-flight and amplitudes may be recorded by the controller 4 using phase, impulse response or other suitable determinations. In one embodiment, the input acoustic signal is a pseudorandom noise signal. The controller 4 then cross-correlates this input signal with the signals received at the receivers 3a,3b, e.g. by cross-correlating the received signals with a signal produced at a receiver near the transducer or by cross-correlating them with the control signal applied to the transducer. The controller 4 may also or alternatively cross-correlate signals received at pairs of receivers, for example, the two signals received at the receivers 3a,3b in FIG. 3.

The cross-correlation can be used to determine the impulse response of the chest, and, by using a Fast-Fourier Transform of this response, the frequency domain transfer function can be determined. Using the FFT, the velocity, attenuation and their dispersion (as a function of frequency) may be determined along with the power spectrum of the lung.

When injecting a pulsed tone signal, the outputs from a plurality of receivers may also be cross-correlated to establish the transit time (velocity) and amplitude of the pulse arriving at the chest wall, at the location of each of the receivers. This process can be repeated for a number of tone frequencies, and using the measurements, a parameter such as velocity dispersion, Δ, may be calculated as:

$$Δ = \frac{{v}_{2} - {v}_{1}}{{f}_{2} - {f}_{1}} ,$$

where v₁ is the sound velocity measured at frequency f₁, and v₂ is the sound velocity measured at frequency f₂.

The frequency dependence of attenuation could be calculated similarly.

These results may be used to determine the existence and stage of COPD. One particular form of COPD which is well suited to this method of detection is emphysema. The various acoustic characteristics may be compared with those of a normal lung to indicate whether there are significant differences and those differences may be taken as an indication of the presence of a feature of COPD. Also, for any particular subject or subject-type, velocity and attenuation standards may be set for indicating emphysema against which the results may then be compared. In particular, such velocity and attenuation standards may be utilized by the processor to determine the likelihood of an asymptomatic subject developing emphysema, to stage the development of COPD (including emphysema), for example as early/middle/advanced stage, and to provide other clinically useful information.

Generally, a higher than expected velocity for a particular frequency may indicate emphysema, due to increased communication between adjacent alveoli, as may a larger than expected velocity dispersion. A higher than expected velocity for a particular frequency or range of frequencies and lower signal attenuation at higher frequencies, may indicate chronic bronchitis.

The results for the various acoustic transmission characteristics may be combined in the assessment so as to reinforce the judgment and/or so as to indicate the degree or stage of COPD. Thus, a velocity increase and a velocity dispersion increase may be used together to indicate the presence of fenestrae and so emphysema. The acoustic transmission characteristics determined using the inventive method and apparatus may also be used in combination with more traditional methods such as spirometry and x-ray methods, where further clinical support for a finding is warranted.

The degree of velocity increase, and velocity dispersion and the like may also be used to determine the degree or stage of COPD, where later stages of the disease correspond with larger fenestrae and therefore larger changes in detected signal velocities and dispersions.

The present detection method recognizes that COPD and in particular, emphysema can be detected by transmitting an acoustic signal through a lung or part thereof, and monitoring the acoustic transmission characteristics which are attributable to features of COPD such as, in the case of emphysema, a microscopic change in the structure of the alveoli. These changes include appearance of fenestrae in the onset and progression of the disease which can be determined by measurable changes in the acoustic permeability of the lung.

This allows for the provision of a simple, inexpensive and easy to use detection apparatus that may be useful in large scale screening programs. Of particular interest is the ability of the system to detect microscopic emphysema, that is early stage emphysema, where the fenestrae are small (i.e. of the order of 10 to 20 micron), or to identify asymptomatic subjects who are likely to develop emphysema. Such detection has particular economic benefit. Large scale screening of subjects, especially smokers, and identifying those who are likely to go on to develop emphysema by detecting acoustic transmission characteristics indicative of microscopic changes (fenestrae) in the lung can encourage early cessation of smoking and other lifestyle changes, and rehabilitation which can delay development of the disease.

In such embodiments, the processor of the inventive apparatus may be configured to output on a display device a COPD risk indicator which gives an indication of a subject's susceptibility or likelihood (percentage risk, for example) of developing COPD. Another application of a screening program is to screen potential participants for an emphysema drug trial. In this case, participants who are determined to have emphysema are considered suitable for the trial and enrolled. Those who are not determined as having emphysema are not suitable and are not enrolled. This screening method has the advantage of minimizing the risk that the trial will produce spurious results due to participation of subjects without emphysema. It also has the potential to reduce the overall cost of the trial, primarily because a smaller amount of the drug will be required, since all of the participants will provide useful results suitable for determining the effectiveness, efficacy and safety of the drug.

To date, drug treatment of COPD has been somewhat non-specific, with chronic bronchitis COPD sufferers and emphysema COPD sufferers being treated, in many cases, with the same class(es) of drug. These include anti-inflammatory drugs, bronchodilators or corticosteroids. This generally occurs where the patient presents with generic symptoms of COPD and the prescribing physician is unable to ascertain if the COPD is manifested as emphysema, chronic bronchitis or even another form of the disease. To improve on this treatment approach, the inventive screening method and apparatus can also be used to screen COPD sufferers to identify those who are candidates for treatment with a class of drug designed to treat emphysema specifically, rather than chronic bronchitis. This has obvious economic benefits, and has the potential to prevent patients from being treated with a useless and potentially harmful drug.

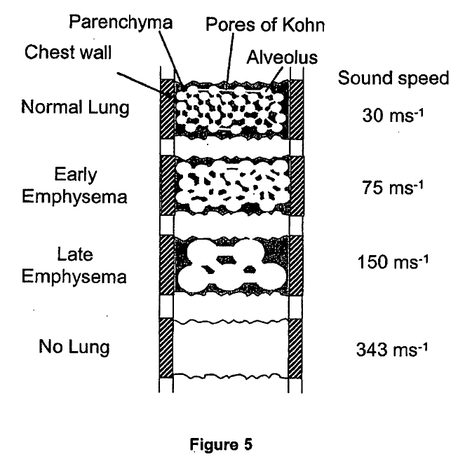

FIG. 5 shows conceptually the change in velocity characteristics with deterioration of the lung. In a normal lung, the speed of sound may be for example, 30 ms⁻¹ for one particular acoustic frequency, and increases with higher frequencies. As the lung deteriorates, however, the number of alveolar fenestrae increases, and the air sacs lose their definition and form larger sacs. As this occurs, the velocity of any particular frequency signal will increase, as shown, for example, to 75 or 150 ms⁻¹, with an extreme limit of no lung tissue (only air) producing a sound wave of 343 ms⁻¹ (which of course will not occur in practice).

From an analytical point of view, an emphysematous lung may be perceived as an elastic material including gas-containing cells that are linked with pores that grow with time as the emphysema progresses. Sound waves propagate through this environment via the pores, which cause a loss of energy via viscous and heat losses to the cell walls. The parameters that determine velocity and attenuation in this setting may be determined by using the conservation laws of mass and momentum that govern wave motion in porous media. These can be stated as follows:

$$\begin{matrix}\frac{\partial {v}_{g}}{\partial x} = - \frac{1}{{K}_{g}} \frac{\partial {p}_{g}}{\partial t} & ( 1 ) \\ \frac{\partial {p}_{g}}{\partial x} = - \frac{{ϕ}_{g} η}{{k}_{0}} {v}_{g} & ( 2 )\end{matrix}$$

where 72, K_{g}, p_{g}, ν_{g} are the viscosity, bulk modulus, pressure and velocity respectively, and φ_{g} is the ratio of gas volume to tissue volume (gas fraction) in the lung, and
k₀ is the permeability or the ease with which sound waves propagate through the porous lung tissue.

Differentiating (2) with respect to x gives:

$$\frac{{\partial }^{2} {p}_{g}}{\partial {x}^{2}} = \frac{{ϕ}_{g} η}{{k}_{0}} \frac{\partial {v}_{g}}{\partial x} \qquad ( 3 )$$

and substituting

$$\frac{\partial {v}_{g}}{\partial x}$$

from (1) into (3) gives:

$$\frac{{\partial }^{2} {p}_{g}}{\partial {x}^{2}} = \frac{{ϕ}_{g} η}{{k}_{0} {K}_{g}} \frac{\partial {p}_{g}}{\partial t} \qquad ( 4 )$$

Transposing (4) gives:

$$\frac{\partial {p}_{g}}{\partial t} = \frac{{k}_{0} {K}_{g}}{{ϕ}_{g} η} \frac{{\partial }^{2} {p}_{g}}{\partial {x}^{2}} \qquad ( 5 )$$

which is a diffusion equation of the form:

$$\frac{\partial {p}_{g}}{\partial t} = {h}^{2} \frac{{\partial }^{2} {p}_{g}}{\partial {x}^{2}} \qquad ( 6 )$$

and has the solution:

$${p}_{g} ( x , t ) = {P}_{0} {ⅇ}^{- \sqrt{ω / 2 {h}^{2}} x} sin ( ω t - \sqrt{ω / 2 h} ) \qquad ( 7 )$$

where ω=2πf is the sound frequency in radians per second,

f is the frequency in Hz, and

P₀ is the sound pressure incident on the lung.

Therefore the wave velocity νₗ and attenuation αₗ in the lung tissue are given by:

$$\begin{matrix}{v}_{l} = \sqrt{2 {h}^{2} ω} = \sqrt{\frac{4 π {k}_{0} {K}_{g} f}{{ϕ}_{g} η}} m / sec & ( 8 ) \\ {α}_{l} = 8.68 \sqrt{ω / 2 {h}^{2}} = 8.68 \sqrt{\frac{π f {ϕ}_{g} η}{{k}_{0} {K}_{g}}} dB / m & ( 9 )\end{matrix}$$

It can be seen from equations (8) & (9) that if K_{g}, η, and φ_{g} are constant, both velocity and attenuation depend on the acoustic permeability of the lung which increases with pore size. It is also evident that the velocity is highly dispersive, increasing as the square root of sound frequency, as does attenuation.

Finally from (8), we can calculate the velocity dispersion with frequency which is:

$$\frac{ⅆ {v}_{l}}{ⅆ f} = \sqrt{\frac{π {k}_{0} {K}_{g}}{f {ϕ}_{g} η}} \qquad ( 10 )$$

Equation 10 indicates that velocity dispersion is directly proportional to the square root of permeability and inversely proportional to the square root of the frequency. Since the current school of thought indicates that permeability of the lung increases with the progression of emphysema, then velocity dispersion would increase over the entire frequency range with development of the disease, but this change is expected to be progressively smaller with increasing frequency. Thus, it is clear that velocity dispersion increases with acoustic permeability of the lung, attributable to an increase in pore size.

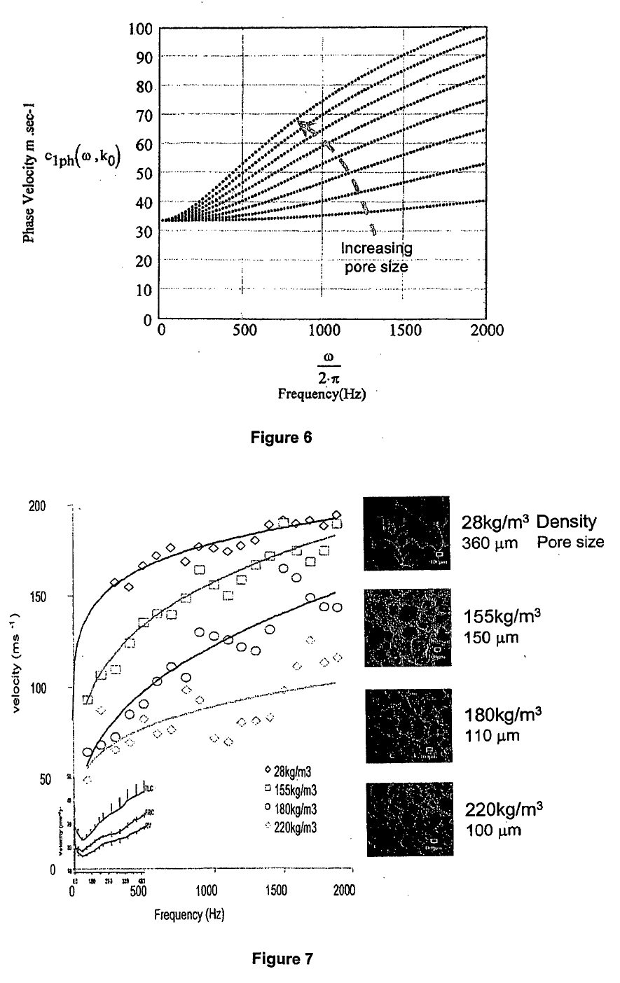

FIG. 6 shows a theoretical graph of frequency versus velocity for various lung permeabilities (permeability being an acoustic parameter that increases as pore size and pore numbers increase). As can be seen, velocities for individual frequencies increase, as does the dispersion (which can be taken as the gradient of the various permeability plots). It is noted here that the acoustic transmission characteristics, e.g. velocity and velocity dispersion, can vary based on both fenestra sizes and the number of fenestrae present in the lung.

FIG. 7 shows the effects on velocity determined using a model of the lung (i.e. a lung analog), in which the pore sizes, i.e. alveolar fenestrae sizes, are increased. As can be seen, both the velocity and velocity dispersion of an acoustic signal increase with increase in permeability. Latex foam and polyurethane foam among other foam materials may be used as emphysematous lung analogs. FIG. 7 also has superimposed on it plots taken from actual subjects having normal lungs at total lung capacity (TLC), functional residual capacity (FRC) and residual volume (RV). The superimposed data illustrates a distinct separation of velocities which is indicative of the change in acoustic transmission characteristics which occurs during development of the disease, when compared with the acoustic transmission characteristics of a normal lung.

Unlike the prior art systems, the present invention uses the porous microstructure of the lung tissue to determine COPD and a stage thereof. This methodology is particularly well suited to detection, staging and monitoring of emphysema, manifested by a change in the quantity and size of pores (fenestrae) in the lung, causing the lung structure to change from what may be considered a closed cell-type to an open cell-type structure in which porous communication between adjacent alveoli increases. This facilitates detection of very early stage emphysema (i.e. when the fenestrae are still microscopic in size, and the patient is still substantially asymptomatic) which hitherto has not been possible using such a non-invasive, easy to use and economical method and apparatus.

It is to be understood that various alterations, additions and/or modifications may be made to the parts previously described without departing from the ambit of the present invention, and that, in the light of the above teachings, the present invention may be implemented in a variety of manners as would be understood by the skilled person.

The present application may be used as a basis for priority in respect of one or more future applications, and the claims of any such future application may be directed to any one feature or combination of features that are described in the present application. Any such future application may include one or more of the following claims, which are given by way of example and are non-limiting with regard to what may be claimed in any future application.

### REFERENCES

Australian and New Zealand Neonatal Network. Annual Report, 1996-1997.

Baumer J H. International randomized controlled trial of patient triggered ventilation in neonatal respiratory distress syndrome. Arch Dis Child 82: F5-F10, 2000.

Bernstein G, Mannino F L, Heldt G P, Callahan J D, Bull D H, Sola A, Ariagno R L, Hoffman G L, Frantz I D 3^{rd}, Troche B I, Roberts J L, Dela Cruz T V, and Costa E. Randomized multicenter trial comparing synchronized and conventional intermittent mandatory ventilation in neonates. J Pediatr 128: 453-63, 1996.

Dreyfuss D, Basset G, Soler P and Saumon G. Intermittent positive-pressure hyperventilation with high inflation pressures produces pulmonary microvascular injury in rats. Am Rev Resp Dis 132: 880-884, 1985.

Fahy, F. (1985) Sound and Structural Vibration. Radiation, Transmission and Response. London: Academic Press.

Froese A B. Role of lung volume in lung injury: HFO in the atelectasis-prone lung. Acta Anaesthesiol Scand Suppl 90:126-130, 1989.

Froese A B. High frequency oscillatory ventilation for adult respiratory distress syndrome: Let's get it right this time! Crit Cae Med 25: 906-908, 1997

Gerstmann D R, Minton S D, Stoddard R A, Meredith K S, Monaco F, Bertrand J M, Battisti O, Langhendries J P, Francois A and Clark R H. The Provo multicenter early high-frequency oscillatory ventilation trial: improved pulmonary and clinical outcome in respiratory distress syndrome Pediatrics. 98: 1044-1057, 1996.

Goncharoff, V., Jacobs, J E, and Cugell, D W Wideband acoustic transmission of human lungs. Med. Biol. Eng. Comp. 27:513-519, 1989.

HIFI Study Group. High frequency oscillatory ventilation compared with conventional mechanical ventilation in the management of respiratory failure in preterm infants. N Engl J Med 320: 88-93, 1989.

Jobe A. Pulmonary surfactant therapy. N Engl J Med 328: 861-864, 1993.

Kraman, S. S. Speed of low-frequency sound through lungs of normal men. J. Appl. Physiol. 55:1862-1867, 1983.

Lowe R D and Robinson B F. A physiological approach to clinical methods. Churchill, London, 1970.

McCulloch, P R, Forkert P G and Froese A B. Lung volume maintenance prevents lung injury during high-frequency oscillatory ventilation in surfactant-deficient rabbits. Am Rev Respir Dis 137: 1185-1192, 1988.

Northway H Q, Rosen R C and Porter D Y. Pulmonary disease following respiratory therapy of hyaline membrane disease. N Engl J Med 276: 357-368, 1967.

Rice, D. A. (1983) Sound speed in pulmonary parenchyma. J. Appl. Physiol. 54:304-308.

Rife D D & Vanderkooy J. Transfer function measurement with maximum length sequences. J Audio Eng Soc 37: 419-444, 1989.

Sheridan, B (2000) Acoustic evaluation of lung inflation in the preterm infant. B. Med. Sci. Thesis, RCBHR, Monash University.

Taghizadeh A & Reynolds EOR. Pathogenesis of bronchopulmonary dysplasia following hyaline membrane disease. Am J Pathol 82: 241-264, 1976.

Wodicka, G. R. and Shannon, D. C. Transfer function of sound transmission in subglottal human respiratory system at low frequencies. J. Appl. Physiol. 69(6):2126-2130, 1990.

Wodicka G R, Stevens, K N, Golub, H L, Cravalho, E G and Shannon, D. C. A model of acoustic transmission in the respiratory system. IEEE Ttrans Biomed. Eng. 36: 925-934, 1989.

1. A method for determining the presence of Chronic Obstructive Pulmonary Disease (COPD) in a lung, the method including the steps of:
applying an acoustic signal to the lung;
detecting the signal after it has passed through at least part of the lung; and
determining an acoustic transmission characteristic indicative of the microstructure of the at least part of the lung;
wherein COPD is determined to be present when the acoustic transmission characteristic indicates the existence of a feature of COPD.

2. The method of claim 1, wherein the acoustic signal is in the frequency range of about 20 Hz to about 25 KHz.

3. The method of claim 1, wherein the acoustic signal is in the frequency range of about 70 Hz to about 5 KHz.

4. The method of claim 1, wherein the acoustic signal frequency is less than 1 KHz.

5. The method of claim 1, further including the step of determining the velocity of the detected signal.

6. The method of claim 5, wherein the signal velocity at a particular signal frequency is used to determine COPD.

7. The method of claim 5 wherein velocity dispersion over a range of acoustic frequencies is used to determine COPD.

8. The method of claim 5 wherein a change in velocity is used to determine COPD.

9. The method of claim 1, further including the step of determining attenuation of the detected acoustic signal.

10. The method of claim 9, wherein the signal attenuation at a particular signal frequency is used to determine COPD.

11. The method of claim 10, wherein the frequency dependence of attenuation over a range of acoustic frequencies is used to determine COPD.

12. The method of claim 1, wherein two or more transmission characteristics selected from the group including but not limited to velocity, velocity dispersion, attenuation, attenuation dispersion and acoustic signal power density and dispersion are used to determine COPD or progression thereof.

13. The method of claim 1, wherein the acoustic signal is applied trans-thoracically.

14. The method of claim 1, wherein the acoustic signal is applied in the region of the supra-clavicular space.

15. The method of claim 1, wherein the acoustic signal is detected at two or more locations on the dorsal spine.

16. The method of claim 15, wherein the acoustic transmission characteristic is determined for the lung region between the two detection locations.

17. The method of claim 15, further including the step of cross-correlating the detected signals.

18. The method according to claim 17 further including the step of applying a Fourier transform to the detected signals.

19. The method of claim 1, wherein the acoustic signal is selected from the group of signals including but not limited to: one or more pure tones applied simultaneously, one or more pure tones applied one after the other, and pseudorandom noise.

20. The method of claim 1, wherein the acoustic transmission characteristic of the lung is determined at functional residual capacity of the lung.

21. The method of claim 1 used to determine a likelihood of an asymptomatic subject developing COPD.

22. The method of claim 1, used to stage progression of COPD in a subject as early, mid or advanced.

23. The method of claim 1 used to determine the existence of COPD in the form of emphysema, wherein an acoustic transmission characteristic indicative of the existence of fenestrae in the at least part of the lung indicates the presence of emphysema.

24. The method of claim 23 used to determine a likelihood of an asymptomatic subject developing emphysema.

25. The method of claim 23 used to stage progression of emphysema in a subject as early, mid or advanced.

26. The method of claim 23 wherein an increase in velocity dispersion is indicative of emphysema.

27. A method for screening participants for an emphysema drug trial according to the method of claim 23, wherein participants determined to have emphysema are considered suitable for the trial.

28. A method, including the steps of claim 23, for determining whether a COPD sufferer is suitable for treatment with a drug for treating emphysema, wherein a COPD sufferer determined to have emphysema is considered suitable.

29. Apparatus for determining the presence of Chronic Obstructive Pulmonary Disease (COPD) in a lung, the apparatus including:
a transmitter for transmitting an acoustic signal into a lung;
one or more receivers for detecting the acoustic signal after it has passed through at least part of the lung; and
a controller/processor for determining an acoustic transmission characteristic indicative of the microstructure of the at least part of the lung and for determining whether the transmission characteristic indicates the presence of a feature of COPD.

30. Apparatus according to claim 29 wherein the one or more receivers are located on the dorsal spine.

31. Apparatus according to claim 29 wherein there are 2 or more receivers located on the torso below the armpit.

32. Apparatus according to claim 30 wherein the receivers are provided in pairs arranged along the dorsal spine.

33. Apparatus according to claim 30 wherein a plurality of receivers are arranged in an array around the dorsal spine.

34. Apparatus according to claim 31 further including a sheath wearable by a subject, the sheath being configured to retain the one or more receivers in position during use of the apparatus.

35. Apparatus according to claim 34 wherein the one or more receivers include hydrophones retained within a fluid-filled cavity of the sheath.

36. Apparatus according to claim 29 further including means to determine a distance between (a) the transmitter and one or more receivers; and/or (b) two receivers.

37. Apparatus according to claim 29 further including a display device for displaying information relating to one or more factors selected from the group including but not limited to the following:
(a) a likelihood of developing COPD;
(b) a likelihood of developing COPD in the form of emphysema;
(c) a presence of COPD;
(d) a presence of COPD in the form of emphysema;
(e) a stage of COPD;
(f) a stage of COPD in the form of emphysema;
(g) suitability of a subject for participating in an emphysema drug trial;
(h) suitability of a subject for treatment with an emphysema drug; and
(i) a map of the existence of COPD in a subject's lung.

38. Apparatus according to claim 29, wherein the transmitter transmits the acoustic signal with a frequency range of about 20 Hz to about 25 KHz.

39. Apparatus according to claim 29, wherein the transmitter transmits the acoustic signal with a frequency range of about 70 Hz to about 5 KHz.

40. Apparatus according to claim 29 wherein the transmitter transmits the acoustic signal with a frequency of less than 5 KHz.

41. Apparatus according to claim 29 wherein the controller/processor is configured to use one or more of the following characteristics to determine presence of COPD in the lung:
(a) velocity of the detected signal;
(b) detected signal velocity at a particular signal frequency;
(c) detected velocity dispersion;
(d) changes in velocity and/or velocity dispersion over time;
(e) acoustic signal attenuation;
(f) acoustic signal attenuation at a particular frequency;
(g) frequency dependence of acoustic signal attenuation;
(h) changes in frequency dependence of acoustic signal attenuation over time;
(i) power distribution over the frequency spectrum of the acoustic signal; and
(j) changes in power distribution over the frequency spectrum of the acoustic signal measured over time.

42. Apparatus according to claim 29 wherein the controller/processor is configured to determine acoustic transmission characteristics for a region of lung located between two receivers.

43. Apparatus according to claim 29 wherein the transmitter transmits an acoustic signal selected from the group including but not limited to: one or more pure tones applied simultaneously, one or more pure tones applied one after the other, and pseudorandom noise.

44. A method for determining chronic obstructive pulmonary disease (COPD), including the steps of: applying an acoustic signal to the lung, measuring the signal after it has passed through the lung, determining an acoustic transmission characteristic of the lung, and determining the presence of chronic obstructive pulmonary disease by determining whether the acoustic transmission characteristic indicates the existence of COPD features, e.g. indicate fenestrae in the alveoli.

45. A method of determining emphysema based on an acoustic transmission characteristic of the lung which assumes a lung structure that has a degree of open-cell structure.

46. A method of determining emphysema based on an acoustic transmission characteristic of the lung, including the step of modeling the lung to have a degree of open-cell structure, and determining an acoustic transmission characteristic associated with such a structure.

47. A method for determining the presence of emphysema in a lung, the method including the steps of: applying an acoustic signal to the lung, detecting the signal after it has passed through at least part of the lung, determining an acoustic transmission characteristic of the lung, and determining the presence of emphysema by determining whether the acoustic transmission characteristic indicates the existence of fenestrae in the alveoli.

48. A method of determining characteristics of biological tissue in situ, including:
introducing a sound to the tissue at first position;
detecting the sound at another position spaced from the first position after it has traveled through the tissue;
calculating the velocity and attenuation of sound that has traveled through the tissue from the first position to another position; and
correlating the velocity and attenuation of the detected sound to characteristics of the biological tissue.
